(19) United States
(12) Patent Application Publication
(10) Pub. No.: US 2016/0015631 A1
(43) Pub. Date: Jan. 21, 2016

(54) OCULAR DRUG DELIVERY SYSTEM

(71) Applicant: Jade Therapeutics, Inc., Park City, UT (US)

(72) Inventor: Barbara Wirostko, Park City, UT (US)

(21) Appl. No.: 14/872,864

(22) Filed: Oct. 1, 2015

Related U.S. Application Data
(62) Division of application No. 13/977,870, filed on Aug. 22, 2013.
(60) Provisional application No. 61/428,085, filed on Dec. 29, 2010.

(51) Int. Cl. A61K 9/00; A61K 47/34; A61K 45/06; A61K 38/27

(52) U.S. Cl. CPC A61K 9/0048; A61K 38/27; A61K 47/34; A61K 45/06

(57) ABSTRACT

An ocular drug delivery system can include a composition in which a formulation including recombinant human growth hormone (rHGH) is contained in a polymer matrix. The composition is configured for placement in or on the eye of a subject, and provides controlled release of an amount of the rHGH to the eye effective to promote healing of a conjunctival, sclera and or corneal wound.

### RELATED APPLICATIONS

This application claims the benefit of U.S. application Ser. No. 13/977,870, filed Aug. 22, 2013, which is a national stage entry under 35 U.S.C. 371 of PCT International Application No. PCT/US11/067942, filed Dec. 29, 2011, which claims priority to U.S. Provisional Patent Application Ser. No. 61/428,085, filed on Dec. 29, 2010, which are each incorporated herein by reference.

### BACKGROUND

Persistent Corneal epithelial defects (PCED) can be defined as a loss of the integrity of the corneal surface and/or a defect in the epithelium, whether caused by injury or disease, which can persist for weeks, months or even years. Corneal stromal ulceration may or may not be associated with PCED. Examples of underlying disease states that may result in such defects include: previous herpes simplex or herpes zoster infections; neurotrophic keratitis after damage to or loss of the fifth cranial nerve function that can be associated with disease states such as diabetes; exposure keratitis secondary lid laxity, position and or closure abnormalities such as Bell's Palsy and aqueous, lipid and or mucin-deficient dry eye states, e.g. occurring after chemical injuries, chronic topical medication use, in patients with Stevens-Johnson syndrome, or in patients with ocular cicatricial pemphigoid. Non-healing corneal epithelial defects may also occur after ocular surgery or other physical injuries to the cornea, and can also result from chronic and overnight contact lens use. These non-healing defects can lead to corneal ulcers, corneal scarring, opacification, and can result in visual loss.

Corneal wound healing and/or re-epithelialization is a highly regulated process that involves the reorganization, migration, and proliferation of epithelial cells from limbal stem cells. Rapid re-epithelialization of the injured area can function in reducing the risk of microbial superinfection, corneal opacification and scarring. Compounds that can accelerate wound closure by increasing the migration and proliferation of the epithelial cells are of interest because of their major potential benefit for patients with epithelial damage such as from dry eye, surgical and non-surgical trauma, refractive interventions, corneal abrasion, non healing corneal ulcers and neurotrophic corneas secondary to diabetes, cranial nerve palsies, and herpetic keratitis. Patients suffering corneal defects can benefit from pharmaco-therapeutic agents that enhance the healing of the cornea through epithelial cell migration.

### SUMMARY

The present technology includes systems that can be used in creating a healing therapy for corneal and ocular surface epithelial defects. In an embodiment, an ocular drug delivery system includes a composition comprising a polymer matrix in which is contained a formulation including recombinant human growth hormone (rHGH). The composition is formulated for delivery to an eye of a subject, and provides controlled release of an effective amount of the rHGH to the eye.

Although a variety of formulation types are contemplated, in one embodiment the composition can be formed as a microparticle suspension, a nanoparticle suspension, a monolithic rod, a gel, a contact lens, or the like. The composition can be further formulated for subconjunctival, subtenons or sub scleral placement, and/or peribulbar, conjunctival cul de sac, or retrobulbar deposit. In another embodiment, the composition can form a sustained release depot. Further, the composition can be injectable. In one embodiment, the polymer matrix can be delivered directly to the target tissue or placed in a suitable delivery device that is either biodegradable and or bioresorbable or can be removed upon completion of the drug delivery.

The composition can provide controlled release of rHGH for an extended duration, e.g. from 4 days to about 200 days. Release of rHGH can further exhibit zero-order kinetics for substantially the entire release duration with a tapering off as the drug substantially completes release. Release modes provided include continuous release and pulsed release. The amount of rHGH released by the depot can be up to zero-order kinetics for substantially the entire duration. In another aspect, the concentration of rHGH in the matrix is from about 0.05 μg to about 100 μg per milliliter. In yet another aspect, the depot provides a total daily concentration of rHGH from about 0.2% to about 2.0%.

The polymer matrix of the delivery composition can include a bioerodible polymer that erodes to provide a rate of controlled release. Such bioerodible polymers that can be used include polyester amides, amino acid based polymers, polyester ureas, polythioesters, polyesterurethanes, collagen based polymers, and copolymers and mixtures thereof. In one embodiment, the bioerodible polymer exhibits an amino acid polymerized via hydrolytically labile bonds at a side chain of the amino acid. In another embodiment, the polymer is a polymerization product of at least one of glycolic acid, glycolide, lactic acid, lactide, e-caprolactone, p-dioxane, p-diozanone, trimethlyenecarbonate, bischloroformate, ethylene glycol, bis(p-carboxyphenoxy) propane, and sebacic acid. In one aspect, glycolic acid and lactic acid are present in a ratio selected to provide a rate of controlled release.

The formulation can be contained in the polymer matrix as a solid, a powder, a gel, or an emulsion. The formulation can further include a second bioactive agent, such as, but not limited to, antibiotics, anti-inflammatory steroids, non-steroidal anti-inflammatory drugs, analgesics, artificial tears solutions, cellular adhesion promoters, growth factors, decongestants, anticholinesterases, glaucoma hypotensive agents, anti angiogenesis drugs (anti VEGFs), antiallergenics, or combinations of any of these. In a particular embodiment, the depot is situated adjacent to a rate controlling diffusion barrier.

A method of making an ocular drug delivery depot, including compositions for the system described above, includes dispersing a formulation including rHGH in a polymer matrix selected to provide controlled release of an amount of the rHGH to the eye.

A method of promoting healing of corneal wound in a subject includes placing a drug delivery composition in an eye of the subject. The drug delivery composition includes a formulation including rHGH contained in a polymer matrix that provides continuous controlled release of an effective amount of the rHGH to the eye, ocular surface and surrounding ocular tissue. In a particular embodiment, placement can be made subconjunctivally, more particularly in subconjunctival locations such as the limbus, the periocular region, sub-Tenon's space, conjunctival cul de sac, sub sclera, sub corneal and the retrobulbar space. In a more particular example, placement of the composition is deliverable by injection. In another embodiment, the composition is placed under or within a contact lens. In still another embodiment a signal can be applied to the drug delivery system after implantation to alter the controlled release. The signal may be a remote signal. In a particular example, the controlled release occurs via iontophoresis.

### BRIEF DESCRIPTION OF THE DRAWINGS

FIG. 1 is an exemplary aqueous SEC-HPLC chromatogram A) release sample containing rHGH; B) release medium. rHGH elutes at approximately 17 min.

FIG. 2 is a bar graph showing bioactivity assay results. Concentration of rHGH in the cell culture medium as determined by SEC-HPLC (light bars). Concentration of active rHGH in the cell culture medium as determined by the cell assay (dark bars). “A” denotes release samples that have been autoclaved prior to introduction to the cell culture medium (negative control). Samples denoted with an asterisk (*): calculated concentrations of “active” rHGH exceed 250 pg/mL.

### DETAILED DESCRIPTION

In describing embodiments of the present invention, the following terminology will be used.

The singular forms “a,” “an,” and “the” include plural referents unless the context clearly dictates otherwise. Thus, for example, reference to “an active agent” can include reference to one or more of such agents and “administering” can include one or more of such administration steps.

As used herein, the term “substantially” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking the nearness of completion will be so as to have the same overall result as if absolute and total completion were obtained.

As used herein, a plurality of items, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “50-250 micrograms” should be interpreted to include not only the explicitly recited values of about 50 micrograms and 250 micrograms, but also to include individual values and sub-ranges within the indicated range. Thus, included in this numerical range are individual values such as 60, 70, and 80 micrograms, and sub-ranges such as from 50-100 micrograms, from 100-200, and from 100-250 micrograms, etc. This same principle applies to ranges reciting only one numerical value and should apply regardless of the breadth of the range or the characteristics being described.

As used herein, the term “about” means that dimensions, amounts, formulations, parameters, and other quantities and characteristics are not and need not be exact, but may be approximated and/or larger or smaller, as desired, reflecting tolerances, conversion factors, rounding off, measurement error and the like and other factors known to those of skill. Further, unless otherwise stated, the term “about” shall expressly include “exactly,” consistent with the discussion above regarding ranges and numerical data.

Human growth hormone (HGH) is a hydrophilic protein with a molecular weight of 22Kda composed of 191 amino acids. HGH is a member of the somatotropin/prolactin family of hormones, and is naturally produced from the pituitary gland. This hormone is required for normal human growth and development. HGH modifies a variety of physiological functions in the body such as, for example, stimulating the expression of insulin-like growth factor I, and increasing calcium retention and bone mineralization. HGH can also increase muscle mass, promote lipolysis, augment wound healing, and reduce liver uptake of glucose. HGH has also been used to treat adults and children for insufficient growth related abnormalities.

It has recently been discovered that HGH can be effective for the treatment of various ocular conditions. It should be noted that any ocular condition that can be treated with a form of HGH is considered to be within the present scope. Additionally, any form of HGH capable of administration to the eye is within the present scope, including naturally produced HGH, synthetic HGH such as recombinant human growth hormone (rHGH), non-human-derived GH, and the like, including combinations thereof. In one aspect, for example, rHGH can be utilized for the treatment of an ocular condition. It is noted that, while the following description refers to rHGH, this is for convenience, and other forms of HGH can be utilized where applicable. Various indications relating to such a condition can include, without limitation, the improved healing of ocular surface defects and various diseases that can result in non-healing ocular defects. Exemplary defects can include delayed corneal wound healing and delayed healing of the sclera and conjunctival epithelium and stroma related to or due to trauma, surgery, systemic and local disease, inflammatory processes, and the like.

It has further been discovered that the application of rHGH in a sustained release manner to the eye can facilitate re-epithelialization of acute and chronic non-healing corneal epithelial defects, conjunctival wounds, and conjunctival and or corneal ulcers, as well as improved healing and nerve reinnervation of diabetic neuropathic corneas and chronic herpetic keratitis. rHGH can also be used to treat recurrent corneal epithelial erosions, severe dry eye with epithelial defects, post surgical corneal defects (i.e. refractive surgery or crosslinking surgery for keratoconus), chemical corneal burns, aseptic corneal perforations, traumatic corneal and conjunctival injuries, and the like.

rHGH can be administered in an immediate effect, sustained release, or a combination of an immediate effect and sustained release formulation, depending on the desired results of a given treatment procedure. In one aspect, rHGH can be formulated and administered as a sustained release ocular delivery system to facilitate longer duration benefits from the hormone. For example, the use of rHGH in a sustained release delivery system can facilitate the resurfacing of an artificial cornea by encouraging the proliferation and migration of endogenous corneal limbal epithelial cells and the regeneration of corneal stromal innervation. Sustained application of rHGH can also provide sufficient growth factor to allow corneal epithelial cell proliferation and migration from grafted epithelial cells, including cells that are derived from pluripotent stem cell grafts and amniotic tissue.

The present technology is directed to systems and methods for sustained delivery of rHGH and other beneficial compounds to the eye of a subject. An ocular drug delivery system in accordance with one embodiment can comprise a composition including rHGH contained in a polymer matrix and formulated for delivery to an eye of a subject. It should be noted that delivery “to an eye” includes delivery onto the surface of the eye as well as delivery into the tissues of the eye. In a particular aspect, the composition provides controlled release of an amount of the rHGH to the eye effective to promote healing of an ocular condition, such as an ocular wound. An ocular wound would include any wound to an ocular tissue surface including, without limitation, the cornea, the sclera, and the palpebral and or bulbar conjunctiva. Also, as used herein in relation to the corneal or scleral surface, “wound” refers to a defect in the cellular structure of the surface, regardless of whether the defect occurred from injury (e.g. corneal trauma, burns, abrasion, and the like such as those due to chemical or blast events), disease, development, human action, etc.

The delivery approach described enables a system including a drug-and-polymer depot to be delivered to the eye such that the active drug is released to the surface of the eye or into tissues of the eye in a continuous or pulsatile manner. As used herein, the term “depot” refers to a collection of material that includes an active agent and that can be placed in an area of interest to provide sustained release of the active agent at least to that area. Accordingly, a method of promoting healing of an ocular wound in a subject can comprise delivering a drug delivery depot as described herein to an eye of the subject. In one embodiment, the depot is placed adjacent to a surface of the cornea, conjunctiva or sclera. Although various placement locations are contemplated, in one aspect placement of the depot can be on or within the sclera (episcleral), beneath or within overlying tissues such as the subconjunctival tissue, e.g. at or near the limbus, within the periocular region, within the conjunctival cul de sac, within the sub-Tenon's space either anterior and or posterior, and in some cases in more posterior retrobulbar locations.

The processes of cell growth and proliferation involved in healing of ocular defects can be ongoing for some time before healing is complete. During that time, the rate and efficiency of these processes can depend on the maintenance of at least a minimum titer of rHGH or other active agent over the healing period. Drug delivery duration may depend upon the severity and underlying process being treated. In one aspect, the composition and location of the composition can be selected to allow the controlled and sustained release of rHGH to occur over a span of from several days to several months. In a specific example, the depot provides controlled release for a period from about 30 days to about 200 days. In another specific example, the depot provides controlled release for a period from about 4 days to about 200 days. In yet another specific example, the depot provides controlled release for a period from about 14 days to about 200 days. In another aspect, the drug-polymer depot can be formulated to provide continuous release having zero-order kinetics over substantially the entire release duration.

Release by the composition provides a dose of rHGH to the eye in which it is placed. In one embodiment, the rHGH is released in a continuous fashion for a particular duration. In an alternative embodiment, the composition provides release of rHGH in a pulsatile fashion, i.e. two or more discrete doses of a given duration and amount and separated by an interval of time. The timing of the pulses can be according to a single fundamental frequency, or can exhibit a more complex temporal pattern. This allows for an additional level of control of release, e.g. to promote greater efficacy or address safety issues. For example, intermittent release can reduce potential adverse effects of continuous HGH stimulation, which may prevent inactivation or down-regulation of receptors. In another example, a pulsatile delivery can be used to simulate and allow a natural course of release of endogenous growth hormone.

Controlled release by the composition can provide to the eye a dose of rHGH that is sufficient to promote healing of corneal defects. In one embodiment, the composition is configured to release a particular amount of rHGH per day. In some embodiments, the composition is configured to release an amount of rHGH sufficient to facilitate the desired effect of the compound in the eye. In another aspect, the composition is configured to release an amount of rHGH that is effective to obtain a desired result. An effective amount or a sufficient amount of rHGH may depend on the type of wound or its etiology. Other possible factors can include the age, weight, medical history of the subject, and the like. Accordingly, the composition can be configured to provide an effective or sufficient dose based on these or other factors. In one embodiment, the composition can provide from about 0.2 mg to about 4.0 mg of rHGH per kg of the subject's body weight. In another embodiment, release of rHGH can be at least 250 mg for a 60 day delivery. In another embodiment, the concentration of rHGH included in the composition polymer material is from about 0.001 mg/ml to about 2 mg/ml. In yet another embodiment, the total concentration in the composition of rHGH can be about 0.2 mg/ml to about 20 mg/ml of solution to polymer. In yet another embodiment, the amount of rHGH provides a concentration of from about 0.001% to about 0.20% rHGH in a 1 ml solution delivered in a 30-50 μl eye drop administered BID-QID. In another embodiment, the total daily concentration delivered of rHGH provided is from about 0.001 mg to upwards of 0.4 mg.

In accordance with one aspect of the present technology, rHGH can be combined with a polymer matrix, and an amount of this combination can be used to create a drug-polymer composition that provides controlled release of rHGH. The physical properties of the composition can be selected to be suitable for different modes of delivery, e.g. topical application, subconjunctival delivery, conjunctival cul de sac, intraocular delivery, transcleral delivery, or the like. It is intended that the present scope include any technique for placing or delivering the composition to the eye, including proximate the eye and/or any portion of the surface sufficient to deliver the rHGH to ocular tissue. Such techniques can include passive delivery techniques, active delivery techniques such as iontophoresis, sonophoresis, and the like. Additionally, the delivery technique can be invasive or non-invasive. Invasive can be defined as any technique whereby a biological membrane is penetrated by a physical object such as a needle during or prior to delivery. Thus microneedle delivery techniques would be considered to be invasive. Accordingly, non-invasive would include any technique whereby a biological membrane is not penetrated by a physical object during delivery. Applying the composition to an exterior eye surface such as, for example, placement into a cul-de-sac and or via a contact lens are examples of passive delivery techniques that are noninvasive. As the drug is released from the polymer matrix, it passively moves into ocular tissue. Iontophoresis is another example of a non-invasive technique.

In one embodiment, the drug-polymer composition can comprise a microparticle or nanoparticle suspension, a solid or semi-rigid monolithic rod, or a gel. In another embodiment, the polymer matrix can be sufficiently liquid to be administered as an eye drop and or topically sprayed as a liquid bandage. In another aspect, the polymer matrix can be injected into an ocular space such as the subconjunctival space. In still another aspect, the drug-polymer matrix can be applied to a structure that is then placed on an ocular surface. Non-limiting examples of such structures include contact lenses, scleral lenses, sponges, polymeric support structures, and the like. With such approaches, the polymer matrix can be selected to be flowable while exhibiting sufficient cohesiveness so that it is not easily diluted or washed away from the placement site. In another embodiment, the polymer matrix itself can be selected and designed to form a supportive structure shaped for placement on or under an ocular surface.

In a particular embodiment, the composition can comprise a polymer matrix that is bioerodible and or bioabsorbable, and can thus be gradually broken down over time, reducing or eliminating the need to remove the polymer matrix at the end of a treatment period. As used herein, “bioerodible” refers to materials that can be broken down by contact with a physiological environment. In many cases such a material can be rendered into smaller pieces that can be further degraded and eliminated by the body. In particular this can refer to rendering the material water-soluble and further resorbable by the body. In one embodiment, controlled release of the active agents from the composition is accomplished by the degradation of bioerodible biopolymers included in the polymer matrix.

In one embodiment, the polymer matrix can include any bioresorbable polymer or mixture of polymers that are compatible with placement in the eye and that can provide the desired release profile. Non-limiting examples of such polymers include, polyester amides, amino acid based polymers, polyester ureas, polythioesters, polyesterurethanes, and the like. In a particular example, bioresorbable polyesters derived from lactone-based biocompatible monomers (glycolide, lactide, e-caprolactone, p-dioxane and trimethlyenecarbonate) can be used. Other possible monomers include bischloroformate, ethylene glycol, bis(p-carboxyphenoxy) propane, and sebacic acid. In a specific embodiment, a bioerodible polymeric composition can comprise a plurality of monomer units of two or three amino acids that are polymerized via hydrolytically labile bonds at their respective side chains rather than at the amino or carboxylic acid terminals by amide bonds. Such polymers are useful for controlled release applications in vivo and in vitro for delivery of a wide variety of biologically and pharmacologically active ligands. According to another embodiment, the polymer matrix can include bioerodible polymers such as polylactic glycolic acid based polymers. Such PLGA polymers can be modified by polycondensation and multiblock copolymers—bischlorofomates, polyethyleneglycol, poly-ε-caprolactone, and the like. By adjusting the lactic/glycolic acid molar ratio in the starting PLGA oligomer, constructs with widely different physicochemical properties can be synthesized through multiblock copolymers. Accordingly, a ratio of glycolic acid and lactic acid can be selected to provide the rate of controlled release. Other suitable polymer matrix materials can include polyesteramides. Such polyesteramides can include alternating diols and di-acids linked by amino acids (commercially available from DSM Biomedical). In particular, dissolution times in aqueous media and in tissue can be tuned within an ample range, from a few days to several months. This provides fine tuning of the polymer device in view of specific applications of delivering biologics to the eye. In the case of multiblock polymers, the nature and the length of the starting diol can be varied to provide the release characteristics such as described above.

Bioerodible ortho ester polymers can also be used for preparing solid form bioerodible pharmaceutical compositions such as pellets, capsules, and rods that can be utilized to contain the rHGH. In a specific example, a bioerodible polyanhydride composed of bis(p-carboxyphenoxy) propane and sebacic acid can also be used as the rHGH carrier for ocular delivery, such as periocular and subconjunctival drug delivery.

In accordance with the present invention, a drug delivery system can utilize other mechanisms for controlled release of an rHGH formulation. For example, in one embodiment the drug-polymer matrix can be substantially contained in a space under a structure on the ocular surface, such as, for example, a contact lens. The composition may be applied to the underside of the contact lens before insertion in the eye, or alternatively the composition can be applied to the cornea and subsequently covered by the lens. In yet another embodiment, the composition can be integrated into a contact lens matrix. For example, the rHGH formulation can be preformed into the lens, adsorbed on the lens surface, or absorbed into the lens polymer. Polymer based contact lenses can be formed with the rHGH in admixture. Alternatively, the contact lens can be immersed in a solution of rHGH for a period of time sufficient to allow a target amount of rHGH to be integrated into the contact lens. Immersion time can be dependent on the contact lens material, temperature, desired target amount and other variables. However, as a general guideline, immersion times can range from about 30 minutes to about 240 minutes. Contact lens polymer materials can include a wide variety of polymers which include, but are not limited to, silicone hydrogel (Alphafilcon A, Asmofilcon A, Balafilcon A, Comfilcon A, Enfilcon A, Etafilcon A, Galyfilcon A, Hilafilcon A, Hilafilcon B, Hioxifilcon A, Hioxifilcon D, Lotrafilcon B, Methafilcon A, Omafilcon A, Phemfilcon A, Polymacon, Senofilcon A, Tetrafilcon A, Vasurfilcon A, Vifilcon A, POLY HEMA, etc.), polymethyl methacrylate, and the like. Upon placement in the eye, the rHGH can then diffuse into the cornea in a delayed release profile.

In another embodiment, the system can include a structure to mediate release of the composition to the eye. In a particular example, the composition can be placed adjacent to a rate controlling diffusion barrier that comprises diffusion control materials, e.g. in a subconjunctival implant and/or within the conjunctival cul de sac. In another example of an implant, release can be aided or accomplished by iontophoresis. The implant can include a membrane or barrier having transport properties that are modulated by changing the electrical state of the barrier. Non-limiting examples of electrically inducible mechanisms for drug release include ion exchange and electroporation. Iontophoretic release can be controlled by application of a signal to the drug delivery system. Such a control signal, e.g. an electrical signal, can be applied directly to the implant, or alternatively can be conveyed by a remote signaling device. To accommodate this type of control, the implant can further include a device, e.g. a microchip, configured to receive and transmit a signal to the barrier that is appropriate to modify the electrical state of the barrier.

In yet another embodiment, the structure can have a hollow interior to contain the rHGH composition and an expanding hydrogel. As the hydrogel expands, the composition is expelled from the structure. The timing of release can be tuned according to the swelling characteristics of the particular hydrogel used.

In yet another embodiment, the structure can be a contact lens having the rHGH contained therein. Thus, the polymer matrix can be formed into a contact lens for direct application to the cornea via the ocular surface. Such polymer matrix may be simply removed upon completion of the treatment or formed of bioerodible polymer as outlined herein. This approach can reduce degradation of the rHGH due to reduced direct contact with enzymes present in tear fluid along the epithelium of the cornea.

In addition to rHGH, the formulation contained in the polymer matrix can include other suitable active agents. The active agents selected can promote wound healing, either independently or in conjunction with the rHGH. Alternatively, active agents having other effects on the condition of the eye can be included. In keeping with the indication of rHGH for wound healing, additional active agents can be chosen that will not interfere with this action of rHGH. Suitable active agents for inclusion can include by way of example:

Antibiotics such as ciprofloxacin, gatifloxicin, moxifloxacin, bacitracin, tobramycin, macrolides, polymyxin, gramidicin, erythromycin, tetracycline, and the like;

Anti-inflammatory steroids such as hydrocortisone, dexamethasone, triamcinolone, prednisolone, fluorometholone, flucinolone acetate, medrysone, and the like, including associated prodrugs;

Non-steroidal anti-inflammatory drugs such as flurbiprofen sodium, diclofenac sodium, ketorolac, indomethacin, ketoprofen, and the like;

Anasthetics such as lidocaine, tetracaine, and the like;

Antifibrotics such as anti TGF beta drugs, TK inhibitors, and the like; and

Growth factors such as, but not limited to, basic fibroblast growth factor, epidermal growth factor, insulin like growth factor, hepatocyte growth factor, neuronal growth factor, brain derived growth factor, and the like.

Other additional active agents can include artificial tears solutions, cellular adhesion promoters, decongestants, anticholinesterases, glaucoma agents, anti-oxidants, cataract inhibiting drugs, antiallergenics, as well as other drugs that may be indicated for use in the eye while not interfering with the action rHGH.

A variety of uses are contemplated and can vary widely depending on the design of the composition, the condition being treated, and other unique factors. As such, the particular uses described should not be seen as limiting. In one particular embodiment, the composition can be configured for a single use, where the polymer matrix and formulation are combined before placement in or on the eye and the composition or implant is removed or degrades upon exhaustion of the formulation. In an alternative embodiment, a formulation can be added to the polymer matrix after implantation, e.g. by injection. Injection can be made through overlying ocular structures (e.g. the conjunctiva in a subconjunctival implantation), or an injection port can be included that provides access to the polymer matrix.

Other options for use of the compositions can include under a scleral flap during glaucoma and or retina surgery and used at the time of refractive surgery. Further, using the compositions with a corneal transplant procedure, or any ocular surgery, optionally in conjunction with explants can be suitable. In addition, the compositions can be implanted with limbal stem cell amniotic graph transplants. Additionally, the device and compositions can be used after a filter trabeculectomy surgery where conjunctival/sclera leak is present and when complications resulting from improper healing of the filter/bleb arise (i.e. over filtration from a conjunctival bleb wound or leak).

### Example

### Preparation of rHGH Containing Construct

A formulation comprised of rHGH and excipients was dispersed into a liquid solution comprised of a polyester amide polymer and organic solvent. Application of appropriate processing methodologies yielded a drug delivery device in which rHGH was contained within a matrix of the polymer.

Release Experimental Details

Polymer devices in which approximately 1-2 wt % rHGH (Creative BioMart; Shirley, N.Y. U.S.A) is contained within the polymer matrix were exposed to predetermined volumes of release medium at 37° C. over set periods of time. The release medium was composed of phosphate buffered saline (pH 7.4) with added bovine serum albumin and penicillin. After a set period time, during which polymer devices were submerged in the release medium and incubated at 37° C., the total volume of release medium was removed with a mechanical pipette and stored at 4° C. for subsequent analysis (i.e., referred to as release samples). A known volume of fresh release medium was then added to the polymer devices with a mechanical pipette and incubation at 37° C. continued. After set periods of time, the release medium was removed and replaced in the same manner as stated above.

Analytical Methods

The concentration of rHGH present in release samples was determined through use of aqueous size exclusion—high performance liquid chromatography (SEC-HPLC). Analysis of release samples were carried out on an Agilent 1200 Series system equipped with a TSKgel G2000SWXL 7.8*300 mm (TOSOH Bioscience) column, Col No 2SWX02SS4835.

Mobile phase: 1.059 mM KH₂PO₄, 2.966 mM Na₂HPO4, 300 mM NaCl, pH=7.4, 10% EtOH (287.16 mg KH₂PO₄, 841.1 mg Na₂HPO₄, 35.64 g NaCl in 2 L Milli-Q water, pH adjusted at 7.4 with NaOH 1N, 222 mL EtOH)

Conditions: Flow 0.5 mL/min for 35 minutes, detection at 220, 250 and 280 nm.

Response factor was calculated from a reference hGH sample (Creative BioMart). rHGH content of this reference was determined by Bradford assay. Response factor was used to calculate the concentration of rHGH in release samples.

Bioactivity of rHGH present in release samples was assessed by measuring its influence on the proliferation of Nb2 (rat lymphoma) cells.

The method used is described below:

Nb2 cells (Sigma-Aldrich) derived from rat T lymphoma cells were cultured in suspension in Fischer's medium supplemented with 10% fetal bovine serum, 10% horse serum, 50 μM 2-mercaptoethanol and 2% penicillin/streptomycin (“culture medium”) in a humidified incubator at 37° C. (5% CO₂). For proliferation assays, cells growing at log-phase were washed two times with the same medium prepared without fetal bovine serum (“incubation medium”) and kept for 24 hours in this medium.

Cells were then counted with a Guava Easycyte (Millipore) capillary cytometer using Viacount reagent (Millipore) to stain cells, according to the recommendations of the manufacturer. The cells suspension was diluted in incubation medium to reach 200.000 viable cells per mL. Cells were plated in 96-well plates (100 μL cell suspension per well).

Samples originating from the release experiments were diluted in incubation medium to reach an expected (according to HPLC quantification) concentration of hGH between 80 and 280 pg/mL (concentration range in which growth of Nb2 cells is hGH concentration-dependent).

These solutions were split in two aliquots and one aliquot was autoclaved. 100 μL of these solutions were added to the Nb2 cells, and cells were incubated for 72 hours at 37° C. After incubation, cells were stained with 50 uL Viacount reagent and viable cells were counted by capillary cytometry.

Results

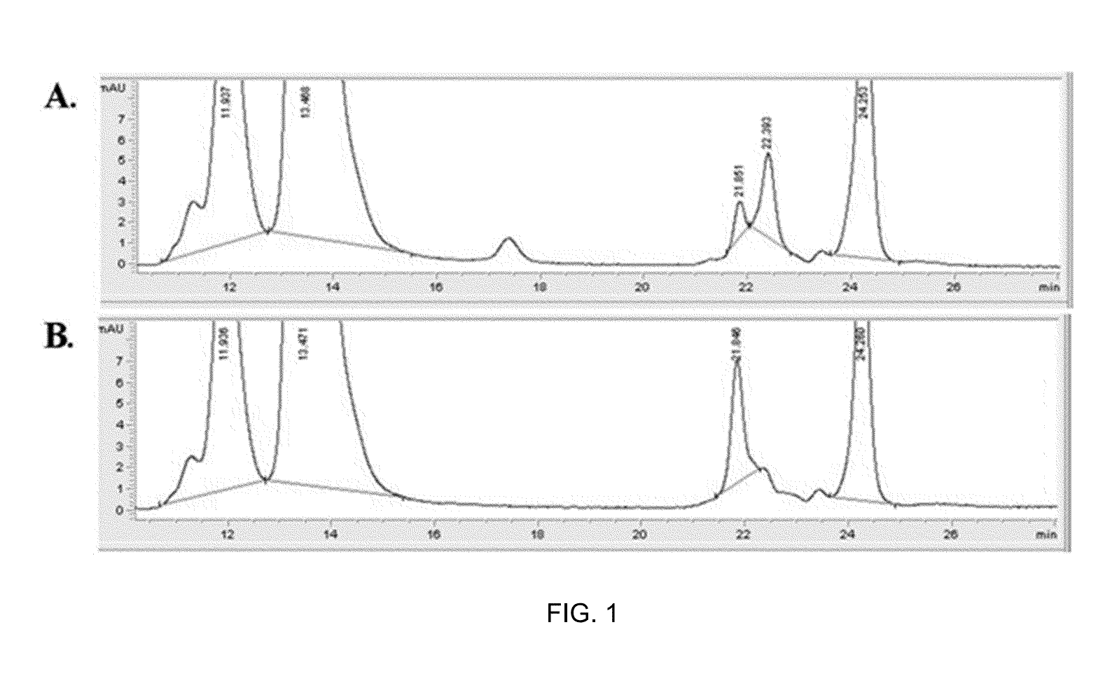

The concentration of rHGH present in release samples at time points from 1 hour to 48 hours was measured using aqueous SEC-HPLC by correlation of peak area (i.e., 17 min elution volume) to rHGH concentration through use of a calibration curve (FIG. 1).

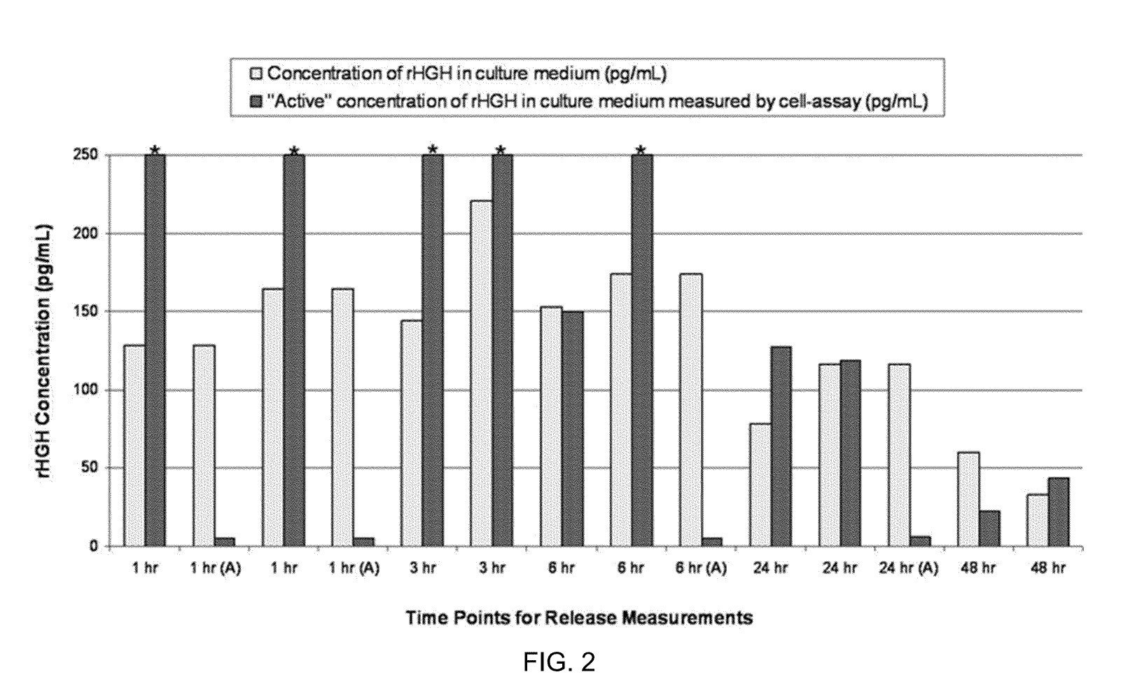

Bioactivity of rHGH released from polymer devices was measured through use of the cell-proliferation assay described above. Release samples were introduced to the cell culture medium and the effect of rHGH on cell proliferation was measured via cell counting with capillary cytometry. A positive cell response was measured from release samples taken at 1 h, 3 h, 6 h, 24 h & 48 h, indicating that rHGH released from the polymer devices was bioactive (FIG. 2). As a negative control, release samples taken at 1 h, 6 h, and 24 h were exposed to elevated temperature and pressure (i.e., autoclave) in order to denature and/or deactivate the rHGH. A qualitative difference in cell response to release samples before and after autoclaving was recorded, validating our experimental method. These results confirm that rHGH present in and subsequently released from the polymer devices is bioactive.

While the examples and details described above are illustrative of the principles of the present invention in one or more particular applications, it will be apparent to those of ordinary skill in the art that numerous modifications in form, usage and details of implementation can be made without the exercise of inventive faculty, and without departing from the principles and concepts of the invention. Accordingly, it is not intended that the invention be limited, except as by the claims.

What is claimed is:

1. A drug delivery system, comprising a composition including recombinant human growth hormone (rHGH) contained in a bioerodable polymer matrix, wherein the matrix does not comprise a microparticle suspension, a nanoparticle suspension or an emulsion, wherein the composition forms a supportive structure shaped for placement on or under an ocular surface and is formulated for delivery to an eye of a subject, and wherein the composition provides controlled release of about 0.001 mg per day to about 0.4 mg per day rHGH to the eye to promote healing of a corneal, scleral or conjunctival condition.

2. The system of claim 1, wherein the composition is formulated for topical ocular delivery.

3. The system of claim 1, wherein the composition is formulated for subconjunctival delivery.

4. The system of claim 3, wherein the composition includes 0.001 mg/ml to 2 mg/ml of rHGH.

5. The system of claim 1, wherein the rHGH has a concentration in the matrix of about 0.05 μg to about 100 μg per milliliter.

6. The system of claim 1, wherein the rHGH has a concentration in the matrix of about 0.2 mg per milliliter to about 2 mg per milliliter.

7. The system of claim 1, wherein the controlled release has a duration of from about 7 days to about 200 days.

8. The system of claim 7, wherein the controlled release exhibits zero-order kinetics for substantially the entire duration.

9. The system of claim 1, wherein the amount of the rHGH is released as a plurality of pulsed doses.

10. The system of claim 1, wherein composition provides a continuous release of the rHGH.

11. The system of claim 1, wherein the bioerodible polymer further comprises a bioerodible polymer selected from the group consisting of polyester amides, amino acid based polymers, polyester ureas, polythioesters, polyesterurethanes, and copolymers and mixtures thereof.

12. The system of claim 1, wherein the bioerodible polymer further comprises an amino acid polymerized via hydrolytically labile bonds at a side chain of the amino acid.

13. The system of claim 1, wherein the bioerodible polymer comprises at least one monomer selected from the group consisting of glycolic acid, glycolide, lactic acid, lactide, e-caprolactone, p-dioxane, p-diozanone, trimethlyenecarbonate, bischloroformate, ethylene glycol, bis(p-carboxyphenoxy) propane, and sebacic acid.

14. The system of claim 13, wherein the at least one monomer includes ethylene glycol.

15. The system of claim 1, wherein the bioerodible polymer includes glycolic acid and lactic acid in a ratio selected to provide the rate of controlled release and the rate of polymer degradation.

16. The system of claim 1, wherein the composition is as at least one of a solid and a gel.

17. The system of claim 1, wherein the formulation further includes a second bioactive agent selected from the group consisting of antibiotics, anti-inflammatory steroids, non-steroidal anti-inflammatory drugs, analgesics, artificial tears solutions, growth factors, decongestants, anticholinesterases, antiglaucoma agents, cataract inhibiting drugs, antioxidants, anti angiogenic drugs, antiallergenics, and combinations thereof.

18. The system of claim 1, wherein the composition is situated adjacent a rate controlling diffusion barrier.

19. A method of promoting healing of a corneal and/or a conjunctival wound in a subject, comprising: placing a drug delivery composition in an eye of the subject, said drug delivery composition comprising a formulation including recombinant human growth hormone (rHGH) contained in a polymer matrix, wherein the polymer matrix provides controlled release of an amount of the rHGH to the eye effective to promote healing.
